(19) United States
(12) Patent Application Publication
(10) Pub. No.: US 2019/0125994 A1
(43) Pub. Date: May 2, 2019

(54) VENTILATORS AND SYSTEMS FOR PERFORMING AUTOMATED VENTILATION PROCEDURES

(71) Applicant: OrangeMed, Inc., Irvine, CA (US)

(72) Inventors: Hong-Lin Du, Irvine, CA (US); Germano Forti, Irvine, CA (US)

(73) Assignee: OrangeMed, Inc., Irvine, CA (US)

(21) Appl. No.: 15/795,839

(22) Filed: Oct. 27, 2017

(51) Int. Cl. A61M 16/00

(52) U.S. Cl. CPC A61M 16/026; A61M 16/0051

(57) ABSTRACT

Examples described herein include systems and ventilators for performing automated ventilation procedures. The automated ventilation procedure may automatically adjust ventilator parameters (e.g., PEEP) to perform a procedure such as a recruitment maneuver, PEEP titration, a recruitability assessment, or combinations thereof.

### BACKGROUND

Patients who cannot breathe by themselves or can only partially breathe by themselves often require mechanical ventilation. These patients include those suffering from pneumonia or acute respiratory distress syndrome (ARDS). In these patients, lungs or alveoli tend to collapse, for example, due to the deficiency of surfactant in the alveoli and lung inflammation, resulting in less effective lung units for gas exchange. One of the goals of mechanical ventilation may be to keep the lungs or alveoli open by recruiting lung units to a maximum possible open level and then by maintaining positive end-expiratory pressure (PEEP) at an ideal level, so as to keep the lung open even during expiratory phase while avoiding high pressure related barotrauma and minimizing negative impact of high airway pressure and high PEEP on circulatory functions. This approach may be referred to as an open lung approach. The process of recruiting lung units may be referred to as a lung recruitment maneuver (RM), which may gradually increase airway pressure step-wise over time, until the lung units are believed to be maximally recruited. The process of finding the optimum PEEP level based on overall lung compliance (or best PEEP level) called PEEP titration (PT), which is achieved by gradually decreasing PEEP step-wise until the PEEP level with the best lung compliance is identified. The PEEP change between each step during PEEP titration may be smaller in order to increase the resolution for the best PEEP level.

### BRIEF DESCRIPTION OF THE DRAWINGS

FIG. 1 is a schematic illustration of a system arranged in accordance with examples described herein.

FIG. 2 is a schematic illustration of a display arranged in accordance with examples described herein.

FIG. 3 is a schematic illustration of a display arranged in accordance with examples described herein.

FIG. 4 is a schematic illustration of a display arranged in accordance with examples described herein.

FIG. 5 is a schematic illustration of a display arranged in accordance with examples described herein.

FIG. 6 is a schematic illustration of a display arranged in accordance with examples described herein.

FIG. 7 is a schematic illustration of a display arranged in accordance with examples described herein.

### DETAILED DESCRIPTION

Certain details are set forth herein to provide an understanding of described embodiments of technology, However, other examples may be practiced without various of these particular details. In some instances, well-known ventilator components, ventilator procedural actions, circuits, control signals, timing protocols, and/or software operations have not been shown in detail in order to avoid unnecessarily obscuring the described embodiments. Other embodiments may be utilized, and other changes may be made, without departing from the spirit or scope of the subject matter presented here.

Historically, ventilator systems have lacked pre-programing for step-wise airway pressure change or step-wise PEEP change, When clinicians perform RM and/or PT manually using a ventilator, the clinicians have generally had to adjust airway pressure or PEEP in a real time manner (e.g. during the ventilator procedure). This makes it difficult and/or impossible for clinicians to simultaneously gather and interpret patient clinical response and tolerance observed through the patient bedside monitor, ventilator or any other source of clinical data, or perform any other care for the patient, during the procedure. Some ventilator systems may continuously (e.g., not step-wise) change airway pressure, rather than PEEP. Continuously changing airway pressure may not provide lung units sufficient time to equilibrate among themselves over a time period such as possible with step-wise changes. Accordingly, the use of continuously changing airway pressure may result in inaccurate measurements of the real lung unit conditions.

Ventilator systems currently available may lack certain safety nets. For example, RM and PT both require the patient airway pressure and PEEP be elevated to much higher levels than what are used during normal ventilation, if the patient uses his own inspiratory muscles to create his own respiratory efforts during these maneuvers, the summation of the patient inspiratory muscle pressure and this high level airway pressure may be so high that it causes barotrauma to the patient lungs. Unfortunately, existing ventilator systems may not provide any alert mechanism to alert clinicians of the existence of the patient's own respiratory efforts, which may leave the patient vulnerable to barotrauma.

Ventilator systems currently available may lack automatic transition between RM and PT. For the patients under mechanical ventilation, often their lungs are not fully recruited during ventilation. Clinicians may want to recruit the patient lungs as fully as possible. At the end of the lung recruitment when the lungs are maximally recruited, PEEP titration may desirably be conducted so as to find the best PEEP level. The best PEEP level can be found retrospectively only after the PEEP is reduced to below the best PEEP level, which causes decruitment of the lung units. Therefore, after the best PEEP level is found retrospectively, the lungs may be re-recruited, after which the ventilator PEEP setting should be set at (or slightly above) the previously measured best PEEP level. Currently available ventilator systems may not provide an automatic transition from RM to PT and to re-recruitment, and they may not automatically set the PEEP to the previously measured best PEEP level upon the completion of re-recruitment.

Examples described herein include a breathing apparatus (e.g., ventilator) system which may provide automated ventilator procedures (e.g., lung recruitment, PEEP titration) using one or more software applications.

Clinicians can accordingly pre-program the steps of airway pressure increase and/or PEEP decrease, the durations for each step, the maximum airway pressure, and maximum and minimwn PEEP. The ventilator may display the predicted time course of the patient airway pressure change and PEEP change, so that clinicians can preview them before the procedures start. When the procedure is in progress, the ventilator may display real-time correlations between PEEP and other parameters such as lung compliance, ventilation volume, blood oxygen saturation, transpulmonary pressure, esophageal pressure, regional alveolar ventilation, circulatory parameters, or combinations thereof.

Examples described herein may provide an alert to alert clinicians of the existence of patient's own respiratory efforts and which may additionally or instead hold or terminate the RM or PT, to minimize the risk of barotrauma to the patient lungs.

Example systems described herein may automatically transition from RM to PT and to RM (re-recruitment), and may automatically set the PEEP (e.g., the PEEP used in the PEEP titration) to a previously measured best PEEP level (e.g., measured during a recruitment maneuver and/or re-recruitment) upon the completion of re-recruitment.

Example systems described herein may automatically calculate a PEEP level that corresponds to the highest lung compliance, lowest transpulmonary pressure, and/or the highest oxygen saturation—this PEEP level may be referred to as a ‘best’ PEEP level.

In some example systems, historical data of correlations between PEEP and the other patient parameters (such as lung compliance, ventilation volume, blood oxygen saturation, transpulmonary pressure, and esophageal pressure) may be retrieved and/or displayed by clinicians, so as to compare such correlations at different measurement times.

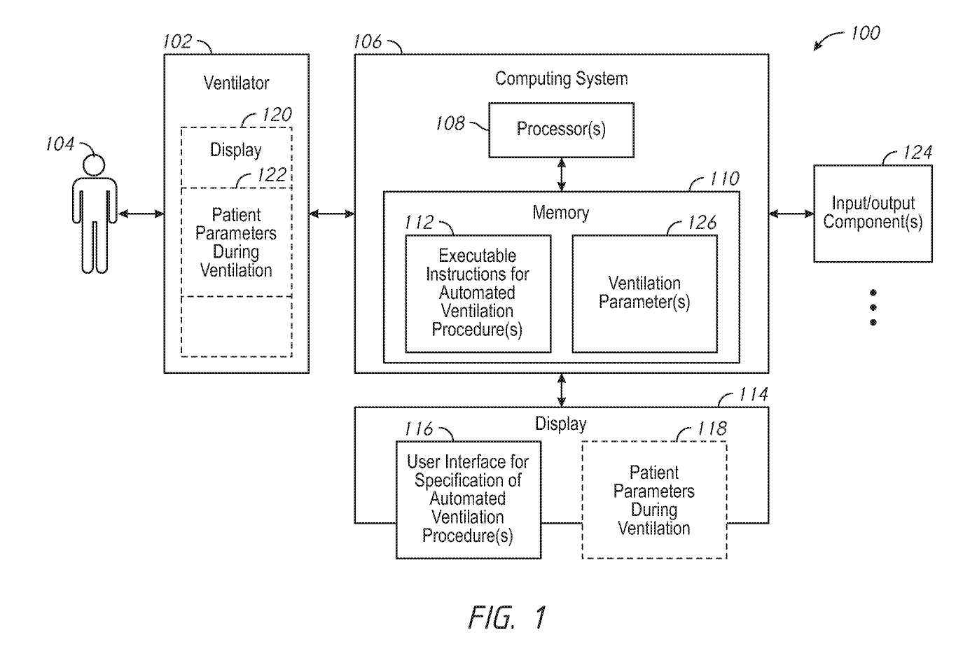

FIG. 1 is a schematic illustration of a system arranged in accordance with examples described herein. System 100 may include ventilator 102, patient 104, computing system 106, processor(s) 108, memory 110, executable instructions for automated ventilation procedure(s) 112, ventilation parameter(s) 126, display 114, user interface for specification of automated ventilation procedure(s) 116, patient parameters during ventilation 118, display 120, patient parameters during ventilation 122, and/or input/output component(s) 124. In other examples, additional, fewer, and/or different components may be used in system 100.

The ventilator 102 may be coupled to patient 104 (e.g. using a breathing tube) to provide mechanical ventilation to patient 104. The ventilator 102 may include display 120 which may in some examples display patient parameters during ventilation 122. The ventilator 102 may be coupled to and/or integral with computing system 106. The computing system 106 may include processor(s) 108 and memory 110. The memory 110 may encode executable instructions for automated ventilation procedure(s) 112 and/or ventilation parameter(s) 126. Execution of executable instructions for automated ventilation procedure(s) 112 may cause the ventilator 102 to perform an automated ventilation procedure. The display 114 may be used to display a user interface for specification of automated ventilation procedure(s) 116 and/or may display patient parameters during ventilation 118. Additional input/output component(s) 124 may be coupled to the computing system 106.

It is to be understood that the arrangement of the components may be quite flexible. For example, although shown separated from the ventilator 102, the computing system 106 may in some examples be integral with the ventilator 102. Although shown as provided in display 114, in some examples, the user interface for specification of automated ventilation procedure(s) 116 may be provided and displayed on a display of the ventilator 102, such as display 120. While shown as stored in a single memory 110, the executable instructions for automated ventilation procedure(s) 112 and/or ventilation parameter(s) 126 may in some examples be stored on separate memor(ies). In some examples, the executable instructions for automated ventilation procedure(s) 112 and/or ventilation parameter(s) 126 may be stored across multiple memory devices.

Examples described herein may include one or more ventilators, such as ventilator 102 of FIG. 1. Any of a variety of ventilators may be used including, but not limited to, mechanical ventilators which operate in assist mode, control mode, provide continuous positive airway pressure (CPAP), provide bi-level positive airway pressure (BPAP), and/or provide high frequency ventilation. Generally, ventilators described herein may assist and/or replace spontaneous breathing by a patient, such as patient 104. The ventilator may move breathable air into and out of lungs of a patient. The ventilator may be connected to a patient using a breathing tube, tracheotomy tube, and/or respiratory mask.

Example ventilators described herein may provide breathable air to a patient, such as patient 104. Any breathing subject may generally be used as a patient in examples described herein, including adult humans, pediatric humans, neonates, and/or animals. Typically, each ventilator may be used to provide breathable air to a single patient. In some examples, however, multiple patients may utilize all or portions of a same ventilator.

Examples described herein may include a computing system, such as computing system 106 of FIG. 1. Any of a variety of devices may be used to wholly or partially implement the computing system 106. in some examples, computing system 106 may be integrated into ventilator 102 (wholly or in part). The computing system 106 may be implemented in some examples using one or more servers, desktops, laptops, computers, tablets, and/or smartphones. Generally, computing systems described herein may be programmed (e.g., include one or more processor(s) and memory which may encode executable instructions, which, when executed cause the computing system to perform certain actions described herein). For example, computing system 106 includes processor(s) 108 and memory 110. Processor(s) described herein may be implemented using one or more processors (e.g., multi-core processors, central processing unit(s)(CPUs), and/or graphics processing unit(s)(GPUs)) and/or specialized circuitry (e.g., application-specific integrated circuits (ASICs), tiled-programmable gate arrays (FPGAs), etc.). memory described herein may generally be implemented using any variety and/or number of computer readable media (e.g., read-only memory (ROM), random access memory (RAM), flash memory, electronic, solid state, optical, and/or magnetic drives, etc.).

Memory described herein may be encoded with executable instructions for performing one or more automated ventilation procedures, such as executable instructions for automated ventilation procedure(s) 112. Generally, the executable instructions for performing one or more automated ventilation procedures may, when executed by the one or more processors, cause a computing system and/or ventilator to perform a ventilation procedure (or a plurality of procedures) without a need for user interaction between a start and an end of the procedure. Examples of a variety of procedures which may be so performed are described herein. The executable instructions for automated ventilation procedure(s) may further include instructions for displaying ventilation data and/or patient parameters during and/or after the ventilation procedure. Moreover, the executable instructions for automated ventilation procedure(s) (and/or other executable instructions in systems described herein) may include instructions for analyzing, combining, and/or manipulating data obtained during and/or after automated ventilation procedures described herein.

Memory described herein may store one or more ventilation parameters, such as ventilation parameter(s) 126. Generally, the automated ventilation procedures described herein may be performed in accordance with one or more of the ventilation parameters. Examples of ventilation parameters include, but are not limited to, starting pressure, maximum pressure, minimum pressure, and/or step size.

Computing systems described herein may be coupled to one or more displays (e.g., may be integrated with one or more displays and/or may have a wired and/or wireless connection to one or more displays), such as display 114 of FIG. 1. The display may be implemented using any of a variety of display technologies, including, but not limited to, liquid crystal display (LCD), light emitter display (LED), plasma, touchscreen, or combinations thereof. Displays described herein, such as display 114 may be used to display a user interface for specification of automated ventilation procedure(s). For example, one or more user interfaces may be displayed which may allow a user to select one or more automated ventilation procedure(s) to perform. Additionally or instead, a user may utilize the user interface to specify one or more ventilation parameters for an automated ventilation procedure. The parameters specified by a user may be stored in a memory, e.g., as ventilation parameter(s) 126 of FIG. 1.

Displays described herein (e.g., display 114 and/or display 120) may display patient parameters during ventilation (e.g., patient parameters during ventilation 122). Patient parameters which may be displayed during ventilation include, but are not limited to, lung compliance, ventilation volume, blood oxygen saturation, transpulmonary pressure, esophageal pressure, or combinations thereof.

Example systems described herein may include any number of input and/or output components (e.g., input/output component(s) 124). The input and/or output components may include such components as keyboard(s), mice, touchscreens, communication interfaces (wired and/or wireless, e.g., Wi-Fi, Bluetooth, USB, and/or HDMI). In some examples one or more of the input/output component(s) 124 may be used to input ventilator parameters through the user interface for specification of automated ventilation procedure(s) 116.

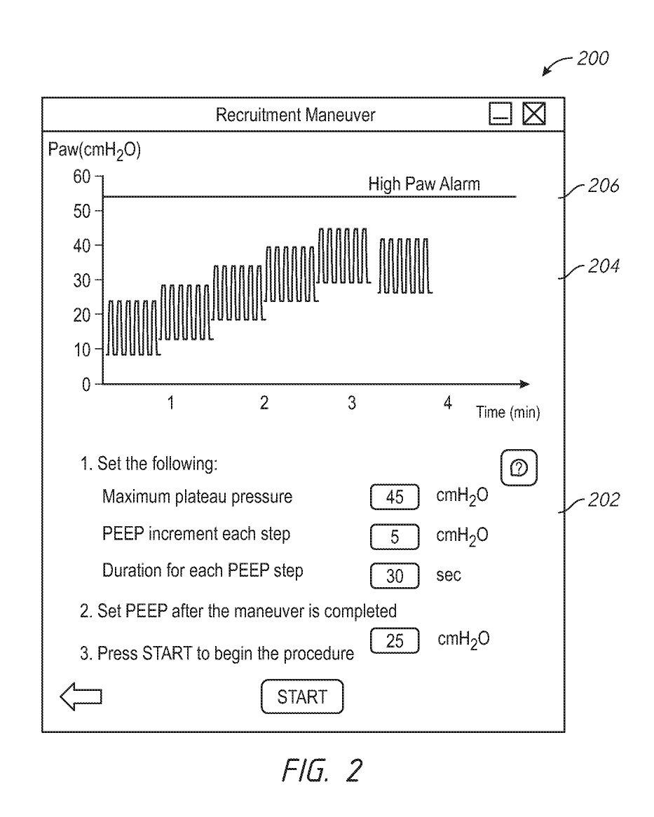

An example of an automated ventilation procedure which may be performed using methods or systems described herein is a recruitment maneuver. FIG. 2 is a schematic illustration of a display arranged in accordance with examples described herein. The display 200 is shown depicting an example of what may be displayed on a user interface (e.g., user interface for specification of automated ventilation procedure(s) 116) when examples described herein are used to perform a recruitment maneuver. The display 114 and/or display 120 of FIG. 1 may be used to implement and/or may be implemented by the display 200 in some examples. Executable instructions for automated ventilation procedures, such as executable instructions for automated ventilation procedure(s) 112 of FIG. 1, may be used to generate all or portions of the information displayed on display 200, and to cause the performance of automated ventilation procedures in accordance with parameters received through the interface shown on display 200.

FIG. 2 depicts user interface for specifying ventilation procedure 202, ventilation procedure preview 204, and alarm level 206. The user interface for specifying ventilation procedure 202 provides a user interface for entry of ventilation parameters which may be used to perform the recruitment procedure. When a user indicates that a particular ventilation procedure is desired (e.g., a recruitment maneuver), then the user interface may display pre-set prompts associated with that particular ventilation procedure to obtain ventilation parameters specific to that particular ventilation procedure. For example, a user may indicate (e.g., by selecting from a menu, touching, typing, or otherwise providing input) that a recruitment maneuver is desired. Responsive to such an indication, the user interface for specifying ventilation procedure 202 may be displayed.

In some examples, prior to displaying the interface shown on display 200 and/or prior to initiating a recruitment maneuver, one or more warnings may be displayed on display 200 (e.g., as specified by executable instructions for automated ventilation procedure(s) 112). The warnings prior to initiating a recruitment maneuver may include, for example, warning that the procedure may cause barotrauma and/or hemodynamic instability. The warning may recommend ensuring suitability of the procedure. A warning may be provided in some examples to ensure the patient is apneic during the procedure. In some examples, while the ventilator may, e.g., utilizing trigger settings, detect patient efforts for respiration and may provide an alert of patient efforts, the ventilator may not deliver triggered breaths (e.g., triggered from patient effort) during the recruitment maneuver. A warning may be provided that the recruitment maneuver is available only for certain modes—e.g., A/CMV PCV or SIMV PCV in some examples. Initial instructions may be displayed on display 200 in some examples for a user—e.g., to select an appropriate mode of the ventilator for the recruitment maneuver, and/or to set one or more appropriate initial ventilation parameters (e.g., set PEEP and/or APC (or PLIMIT) appropriate for a patient and in such a way that ΔPC or inflation pressure (e.g., PLIMIT−PEEP) is between 10-20 cmH2O in some examples.

The user interface for specifying ventilation procedure 202 includes prompts for a user to enter ventilation parameters which may be used in a recruitment maneuver—maximum plateau pressure, PEEP (positive end-expiry airway pressure) increment per step, and duration for each step. The user interface for specifying ventilation procedure 202 as shown further requests a user to input a PEEP for after the maneuver is completed. The user interface for specifying ventilation procedure 202 further includes a button for a user to start the procedure.

Examples of entered user values are shown in FIG. 2—45 cmH₂O maximum plateau pressure, 5 cmH₂O PEEP increment each step, 30 second duration for each PEEP step, and 25 cmH₂O PEEP pressure for after the maneuver is completed. Other values may be used in other examples. In some examples, the executable instructions for automated ventilation procedure(s) 112 may enforce certain ranges for one or more of the parameters. For example, maximum plateau pressure may be enforced within a range of 30-55 cmH2O. PEEP step increment may be enforced to be within a range of 2-5 cmH₂O. Step duration may be enforced to be within a range of 20-120 sec. PEEP after the maneuver may be enforced to be within a range of 0-30 cmH₂O. If a user attempts to enter a value for a parameter outside the predetermined range (note ranges may be stored, for example in memory 110 of FIG. 1), then the display 200 may display an alert regarding the out-of-range parameter, or the parameter may in some other way be rejected by the system. In some examples, parameter values on the display 200 may default to predetermined values. For example, maximum plateau pressure may have a default value of 40 cmH₂O. PEEP step increment may have a default value of 5 cmH₂O. PEEP step duration may have a default value of 30 seconds. PEEP after the maneuver may have a default value of 25 cmH₂O. The default values may be stored, for example, in memory 110 of FIG. 1). The default values may be displayed on display 200 when a user initially selects to perform a recruitment maneuver. The user may then need only change the parameters for which the defaults are not desired. Other ranges and/or default values may be used in other examples. If a user attempts to initiate the recruitment maneuver without a prerequisite being met (e.g., appropriate ventilation mode, appropriate initial settings, in-range ventilation parameters provided), the system may provide an alert and may not initiate the automated recruitment procedure.

Once a user enters the requested ventilation parameters in through the user interface (e.g., by typing, touching, entering, gesturing, speaking, or otherwise providing input), the ventilation parameters may be stored, as ventilation parameter(s) 126 in FIG. 1, The input ventilation parameters may then be used to perform the recruitment maneuver when the user indicates to begin the maneuver (e.g., by clicking, touching or otherwise providing a start input).

As shown in FIG. 2, the display 200 may include a ventilation procedure preview 204. The preview may provide a graphical view of the expected actions of the ventilator responsive to the entered ventilation parameters.

Generally, examples described herein may provide a recruitment maneuver by providing tidal breaths to a patient beginning at a starting PEEP pressure for a step duration specified by the user. After the step duration, the PEEP pressure may be incremented by a PEEP increment specified by a user to obtain a next pressure. Tidal breaths at the next pressure may be provided to a patient for the step duration specified for the user. This process may be repeated until the maximum plateau pressure specified by the user is attained.

Once the recruitment maneuver has attained the maximum pressure specified by the user, the recruitment procedure may be completed, and the ventilator may revert to providing the PEEP pressure specified by the user for after the procedure.

The ventilation procedure preview 204 displays a graph of pressure vs. time that displays the expected ventilator performance for the maneuver. The ventilation procedure preview 204 may display the expected ventilation procedure using the ventilation parameters entered by a user in user interface for specifying ventilation procedure 202. A user may graphically review this performance prior to initiating the automated procedure (e.g., prior to pressing ‘staff’). in some examples, the user may revise the parameters entered in the user interface for specifying ventilation procedure 202 after review of the ventilation procedure preview 204.

In some examples, the executable instructions for automated ventilation procedures described herein may include instructions for providing one or more alarms. The ventilation procedure preview 204 shown in FIG. 2 illustrates an example of an alarm condition. For example, an alarm level 206 may be set such that if a pressure exceeds the alarm level 206, the ventilator and/or computing system will provide an alarm (e.g., an audible, visual, and/or tactile alarm). The alarm level 206 may be displayed in the ventilation procedure preview 204. In some examples, the alarm level 206 may be set by a user, however in some examples the alarm level 206 may be specified by the executable instructions for automated ventilation procedure(s) themselves. Alarms may provide additional safety in some examples.

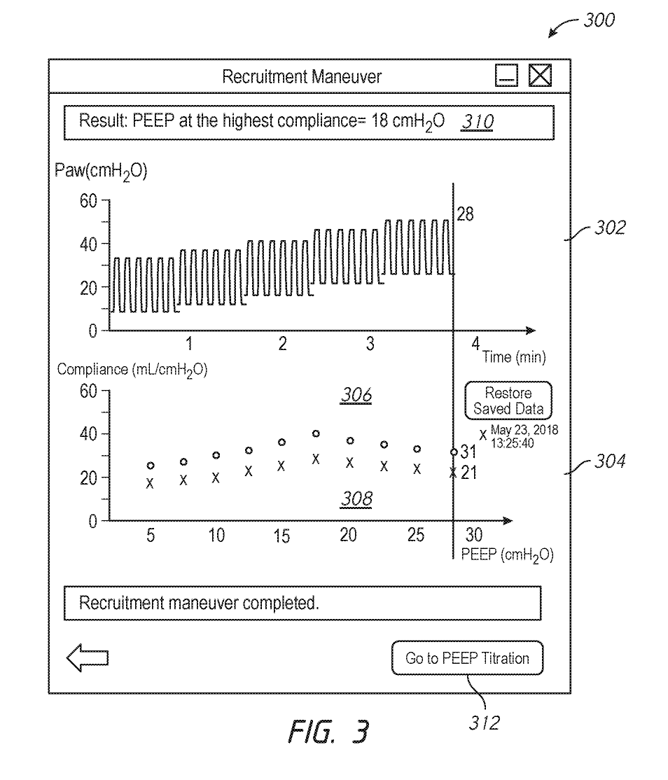

FIG. 3 is a schematic illustration of a display arranged in accordance with examples described herein. Display 300 is shown displaying ventilator data 302, patient parameter data 304, current patient data 306, historical patient data 308, result 310, and button 312. In other examples, additional, fewer, and/or different elements may be displayed. The display 300 may be used to implement and/or may be implemented by display 114, display 120, and/or display 200 in some examples. Display 300 is shown displaying information that executable instructions described herein (e.g., executable instructions for automated ventilation procedure(s) 112 of FIG. 1) may cause to display during and/or after a recruitment maneuver.

During an automated ventilation procedure, such as a recruitment maneuver, display 300 may display ventilator data 302—e.g., data regarding one or more ventilator parameters. Data may be displayed as the ventilator performs the procedure, and may provide information regarding progress of the procedure (e.g., the data may be displayed in real-time). For example, as shown in FIG. 3, a graph of pressure over time is depicted as ventilator data 302. The graph may be dynamic in the display 300 in that it is drawn as the procedure progresses, so that a user may view how the procedure is progressing. While a graph is shown, any of a variety of data display types may be used (e.g., line graph, bar graph, pie graph, text list). Generally, one or more correlations between ventilator data and time or other parameter may be displayed.

During the automated ventilation procedure, such as a recruitment maneuver, display 300 may additionally or instead display patient parameter data 304—e.g., data regarding one or more patient parameters. Any of a variety of patient parameters may be displayed, including, but not limited to lung compliance, ventilation volume, blood oxygen saturation, transpulmonary pressure, esophageal pressure, or combinations thereof. In FIG. 3, the display 300 is shown displaying a graph of lung compliance vs. PEEP pressure. The measured parameter (e.g., lung compliance) may similarly appear in the display 300 dynamically, such that the graph is drawn over time (e.g., in real-time) and a user may view how the patient parameter is progressing during the procedure. Generally, one or more correlations between patient data and one or more ventilator parameter (e.g., pressure) may be displayed.

In some examples, ventilator data 302 and patient parameter data 304 may be depicted on a same display, e.g., display 300, during an automated ventilation procedure. This may advantageously allow a user to review both the progress of the automated ventilation procedure and the patient parameter.

In some examples, in addition to displaying the patient parameter data 304 collected during a current automated ventilation procedure (e.g., current patient data 306), the display 300 may display historical patient data (e.g., historical patient data 308). The historical patient data may be data from the same patient during a similar previous ventilation procedure. In some examples, the historical patient data 308 may be data from another patient, or from a combination of patients (e.g., an average for a particular patient population and/or an average for a particular ventilation procedure). The historical patient data 308 may be displayed all at once as shown, or may also be dynamically displayed over time. In this manner, a user may compare current patient data to historical data as a ventilation procedure is occurring.

During the automated ventilation procedure (e.g., the recruitment maneuver), the system (e.g., system 100 in accordance with executable instructions for automated ventilation procedure(s) 112) may provide an alert if a patient's own respiratory efforts are detected. The alert may be provided, for example, by computing system 106 and/or ventilator 102 of FIG. 1. The alert may be audible, visual, and/or tactile in sonic example. The alert may be provided, for example, on display 300 and/or display 114 and/or display 120. In some examples, the system (e.g., computing system 106 of FIG. 1) may halt the automated ventilation procedure responsive to an alert of a patient's respiratory efforts.

In some examples, the display 300 may display one or more results or other data analysis outputs, e.g., result 310. For example, the executable instructions for automated ventilation procedure(s) 112 may include instructions for analyzing data generated during one or more ventilation procedures, such as a recruitment maneuver. In the example of FIG. 3, the result 310 reported is a PEEP at the highest patient compliance value during the recruitment maneuver—18 cmH₂O in the example of FIG. 3. In this manner, a PEEP at which a patient has a highest lung compliance may be reported in an automated manner. Generally, in examples described herein, results may include one or more ventilation parameters (e.g., PEEP) reported at a particular patient criteria (e.g., highest lung compliance, a lowest transpulmonary pressure, a highest oxygen saturation, or combinations thereof during the positive end-expiratory pressure titration). As described with reference to FIG. 2 and FIG. 3, a user may enter ventilator parameters, start the automated procedure, and then be presented with a result, such as the PEEP at which the patient has the highest lung compliance. The result may be used in a number of ways, including for subsequent treatment of a patient. For example, the PEEP reported by result 310 may be used to ventilate the patient after the recruitment maneuver is complete.

In some examples, a next automated procedure may be suggested by systems described herein. User interfaces provided herein may provide an option to perform the suggested next automated procedure. For example, in FIG. 3, following completion of a recruitment maneuver, a PEEP titration procedure may be suggested. A user may elect to begin the PEEP titration procedure by selecting (e.g., tapping, touching, clicking) button 312. The next automated ventilation procedure may, in some examples, utilize results from a previous automated ventilation procedure (e.g., result 310).

Another example of an automated ventilation procedure which may be performed using methods or systems described herein is a PEEP titration. In some examples, the PEEP titration may be performed (automatically, or by user indication) after a recruitment maneuver. In sonic examples, however, a user may select to perform a PEEP titration without first performing a recruitment maneuver. FIG. 3 is a schematic illustration of a display arranged in accordance with examples described herein. The display 300 is shown depicting an example of what may be displayed on a user interface (e.g., user interface for specification of automated ventilation procedure(s) 116) when examples described herein are used to perform a PEEP titration. The display 114 and/or display 120 of FIG. 1 may be used to implement and/or may be implemented by the display 400 in some examples. Executable instructions for automated ventilation procedures, such as executable instructions for automated ventilation procedure(s) 112 of FIG. 1, may be used to generate all or portions of the information displayed on display 400, and to cause the performance of automated ventilation procedures in accordance with parameters received through the interface shown on display 400.

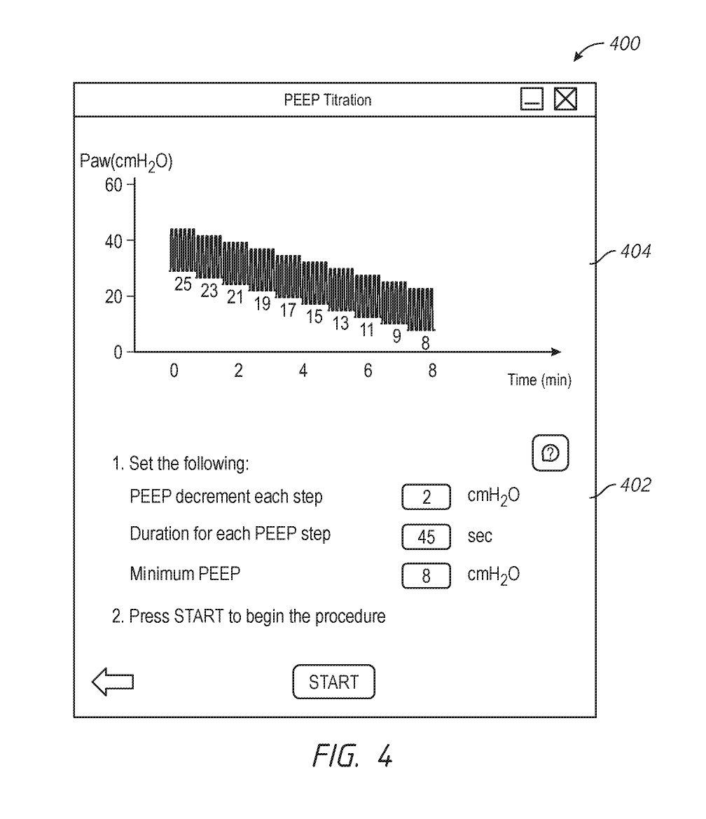

FIG. 4 depicts user interface for specifying ventilation parameter(s) 402 and ventilation procedure preview 404. The user interface for specifying ventilation parameter(s) 402 provides a user interface for entry of ventilation parameters which may be used to perform the PEEP titration. For example, a user may indicate (e.g., by selecting from a menu, touching, typing, or otherwise providing input) that a PEEP titration is desired. Responsive to such an indication, the user interface for specifying ventilation parameter(s) 402 may be displayed.

In some examples, prior to displaying the interface shown on display 400 and/or prior to initiating a PEEP titration, one or more warnings may be displayed on display 400 (e.g., as specified by executable instructions for automated ventilation procedure(s) 112). The warnings prior to initiating a PEEP titration may include, for example, warning that the procedure may cause barotrauma and/or hemodynamic instability. The warning may recommend ensuring suitability of the procedure. A warning may be provided in some examples to ensure the patient is apneic during the procedure. In some examples, while the ventilator may, e.g,, utilizing trigger settings, detect patient efforts for respiration and may provide an alert of patient efforts, the ventilator may not deliver triggered breaths (e.g., triggered from patient effort) during the PEEP titration. A warning may be provided that the PEEP titration is available only for certain modes—e.g., PCV or VCV mode for PEEP titration in some examples. Initial instructions may be displayed on display 400 in some examples for a user—e.g., to select an appropriate mode of the ventilator for the PEEP titration.

The user interface for specifying ventilation parameter(s) 402 includes prompts for a user to enter ventilation parameters which may be used in a PEEP titration—PEEP decrement each step, duration for each step, and minimum PEEP. The user interface for specifying ventilation parameter(s) 402 further includes a button for a user to start the procedure. While buttons and text entry boxes are shown in FIG. 4 and in other user interface figures described herein, it is to be understood that other user interface elements may be used in other examples (including voice control in some examples).

Examples of entered user values are shown in FIG. 4—2 cmH₂O PEEP decrement each step, 45 sec duration for each PEEP step, and 8 cmH₂O minimum PEEP. Other values may be used in other examples. In some examples, the executable instructions for automated ventilation procedure(s) 112 may enforce certain ranges for one or more of the parameters. For example. PEEP decrement each step may be enforced within a range of 1-3 cmH2O. Step duration may be enforced to be within a range of 30-120 seconds. Minimum PEEP may be enforced to be within a range of 0-20 cmH₂O. If a user attempts to enter a value for a parameter outside the pre-determined range (note ranges may be stored, for example in memory 110 of FIG. 1), then the display 400 may display an alert regarding the out-of-range parameter, or the parameter may in some other way be rejected by the system. In some examples, parameter values on the display 400 may default to predetermined values. For example, PEEP decrement each step may have a default value of 2 cmH₂O. Step duration may have a default value of 45 seconds. Minimum PEEP may have a default value of 8 cmH₂O. The default values may be stored, for example, in memory 110 of FIG. 1). The default values may be displayed on display 400 when a user initially selects to perform a PEEP titration. The user may then need only change the parameters for which the defaults are not desired, Other ranges and/or default values may be used in other examples. If a user attempts to initiate the PEEP titration without a prerequisite being met (e.g., appropriate ventilation mode, appropriate initial settings, in-range ventilation parameters provided), the system may provide an alert and may not initiate the automated PEEP titration.

Once a user enters the requested ventilation parameters in through the user interface (e.g., by typing, touching, entering, gesturing, speaking, or otherwise providing input), the ventilation parameters may be stored, as ventilation parameter(s) 126 in FIG. 1. The input ventilation parameters may then be used to perform the PEEP titration when the user indicates to begin the maneuver (e.g., by clicking, touching or otherwise providing a start input).

As shown in FIG. 4, the display 400 may include a ventilation procedure preview 404. The preview may provide a graphical view of the expected actions of the ventilator responsive to the entered ventilation parameters.

Generally, examples described herein may provide a PEEP titration by providing tidal breaths to a patient beginning at a starting PEEP pressure for a step duration specified by the user. After the step duration, the PEEP pressure may be decremented by a PEEP decrement specified by a user to obtain a next pressure. At the transition from one PEEP level to the next PEEP level, an alert (e.g., a message or audio sound) may be used to inform clinicians or other users or programs of such a level change. Tidal breaths at the next pressure may be provided to a patient for the step duration specified for the user. This process may be repeated until the minimum pressure specified by the user is attained.

Once the PEEP titration procedure has attained the minimum pressure specified by the user, the PEEP titration may be completed.

The ventilation procedure preview 404 displays a graph of pressure vs. time that displays the expected ventilator performance for the maneuver. The ventilation procedure preview 404 may display the expected ventilation procedure using the ventilation parameters entered by a user in user interface for specifying ventilation parameter(s) 402. A user may graphically review this performance prior to initiating the automated procedure (e.g., prior to pressing ‘start’). in some examples, the user may revise the parameters entered in the user interface for specifying ventilation parameter(s) 402 after review of the ventilation procedure preview 404.

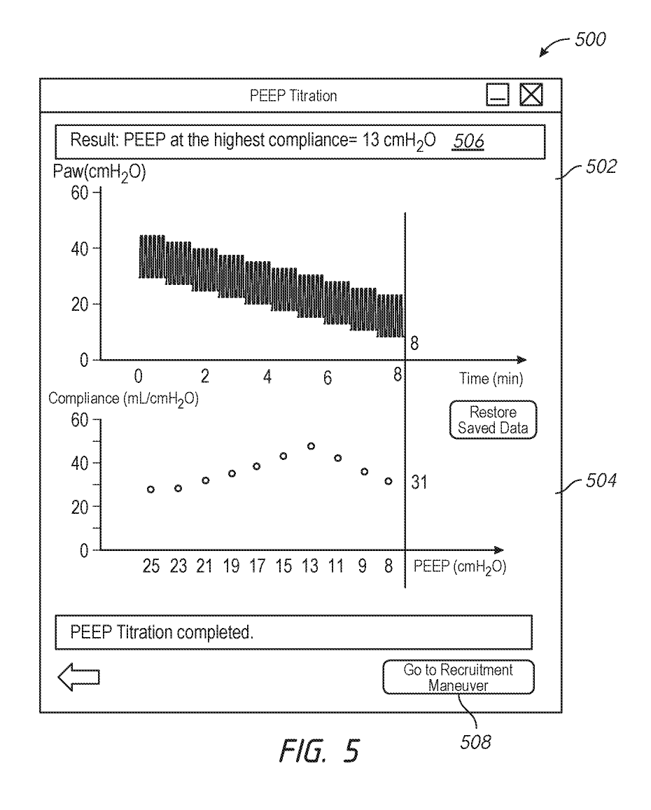

FIG. 5 is a schematic illustration of a display arranged in accordance with examples described herein. Display 500 is shown displaying ventilator data 502, patient data 504, result 506, and button 508. In other examples, additional, fewer, and/or different elements may be displayed. The display 500 may be used to implement and/or may be implemented by display 114, display 120, and/or display 400 in some examples. Display 500 is shown displaying information that executable instructions described herein (e.g., executable instructions for automated ventilation procedure(s) 112 of FIG. 1) may cause to display during and/or after a PEEP titration.

During an automated ventilation procedure, such as a PEEP titration, display 500 may display ventilator data 502—e.g., data regarding one or more ventilator parameters. Data may be displayed as the ventilator performs the procedure, and may provide information regarding progress of the procedure (e.g., the data may be displayed in real-time). For example, as shown in FIG. 5, a graph of pressure over time is depicted as ventilator data 502. The graph may be dynamic in the display 500 in that it is drawn as the procedure progresses, so that a user may view how the procedure is progressing. While a graph is shown, any of a variety of data display types may be used (e.g., line graph, bar graph, pie graph, text list). Generally, one or more correlations between ventilator data and time or other parameter may be displayed.

During the automated ventilation procedure, such as a PEEP titration, display 500 may additionally or instead display patient data 504—e.g., data regarding one or more patient parameters. Any of a variety of patient parameters may be displayed, including, but not limited to lung compliance, ventilation volume, blood oxygen saturation, transpulmonary pressure, esophageal pressure, or combinations thereof. In FIG. 5, the display 500 is shown displaying a graph of lung compliance vs. PEEP pressure. The measured parameter (e.g., lung compliance) may similarly appear in the display 500 dynamically, such that the graph is drawn over time (e.g., in real-time) and a user may view how the patient parameter is progressing during the procedure. Generally, one or more correlations between patient data and one or more ventilator parameter (e.g., pressure) may be displayed. While not shown in FIG. 5, historical patient data may also be displayed during PEEP titrations described herein.

In some examples, ventilator data 502 and patient data 504 may be depicted on a same display, e.g., display 500, during an automated ventilation procedure. This may advantageously allow a user to review both the progress of the automated ventilation procedure and the patient parameter.

In some examples, in addition to displaying the patient data 504 collected during a current automated ventilation procedure, the display 500 may display historical patient data. The historical patient data may be data from the same patient during a similar previous ventilation procedure. In some examples, the historical data. may be data. from another patient, or from a combination of patients (e.g., an average for a particular patient population and/or an average for a particular ventilation procedure). The historical data may be displayed all at once, or may also be dynamically displayed over time. In this manner, a user may compare current patient data to historical data as a ventilation procedure is occurring.

During the automated ventilation procedure (e.g., the PEEP titration), the system (e.g., system 100 in accordance with executable instructions for automated ventilation procedure(s) 112) may provide an alert if a patient's own respiratory efforts are detected. The alert may be provided, for example, by computing system 106 and/or ventilator 102 of FIG. 1. The alert may be audible, visual, and/or tactile in some example. The alert may be provided, for example, on display 500 and/or display 114 and/or display 120. In some examples, the system (e.g., computing system 106 of FIG. 1) may halt the automated ventilation procedure responsive to an alert of a patient's respiratory efforts.

In some examples, the display 500 may display one or more results or other data analysis outputs, e.g., result 506. For example, the executable instructions for automated ventilation procedure(s) 112 may include instructions for analyzing data generated during one or more ventilation procedures, such as a PEEP titration. In the example of FIG. 5 the result 506 reported is a PEEP at the highest patient compliance value during the PEEP titration—13 cmH₂0 in the example of FIG. 5. In this manner, a PEEP at which a patient has a highest lung compliance may be reported in an automated manner. Generally, in examples described herein, results may include one or more ventilation parameters (e.g., PEEP) reported at a particular patient criteria (e.g., highest lunch compliance, a lowest transpulmonary pressure, a highest oxygen saturation, or combinations thereof during the positive end-expiratory pressure titration). As described with reference to FIG. 4 and FIG. 5, a user may enter ventilator parameters, start the automated PEEP titration, and then be presented with a result, such as the PEEP at which the patient has the highest lung compliance. The result may be used in a number of ways, including for subsequent treatment of a patient. For example, the PEEP reported by result 506 may be used to ventilate the patient after the PEEP titration is complete.

In some examples, a next automated procedure may be suggested by systems described herein. User interfaces provided herein may provide an option to perform the suggested next automated procedure. For example, in FIG. 5, following completion of a PEEP titration, a recruitability assessment may be suggested. A user may elect to begin the recruitability assessment by selecting (e.g., tapping, touching, clicking) button 508. The next automated ventilation procedure may, in some examples, utilize results from a previous automated ventilation procedure (e.g., result 506).

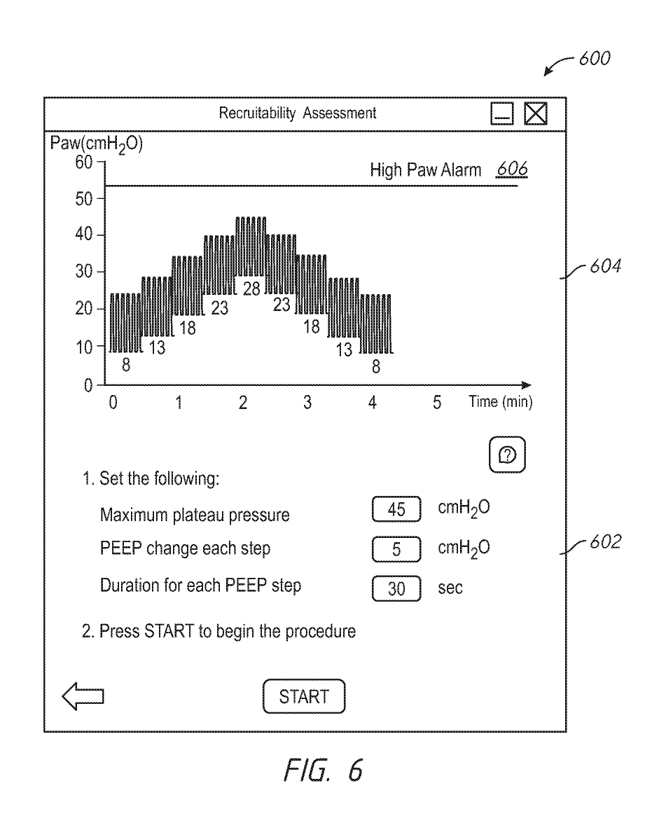

An example of an automated ventilation procedure which may be performed using methods or systems described herein is a recruitability assessment. FIG. 6 is a schematic illustration of a display arranged in accordance with examples described herein. The display 600 is shown depicting an example of what may be displayed on a user interface (e.g., user interface for specification of automated ventilation procedure(s) 116) when examples described herein are used to perform a recruitability assessment. The display 114 and/or display 120 of FIG. 1 may be used to implement and/or may be implemented by the display 600 in some examples. Executable instructions for automated ventilation procedures, such as executable instructions for automated ventilation procedure(s) 112 of FIG. 1, may be used to generate all or portions of the information displayed on display 600, and to cause the performance of automated ventilation procedures in accordance with parameters received through the interface shown on display 600.

FIG. 6 depicts user interface for specifying ventilation procedure 602, ventilation procedure preview 604, and alarm level 606. The user interface for specifying ventilation procedure 602 provides a user interface for entry of ventilation parameters which may be used to perform the recruitability assessment. When a user indicates that a particular ventilation procedure is desired (e.g., a recruitability assessment), then the user interface may display pre-set prompts associated with that particular ventilation procedure to obtain ventilation parameters specific to that particular ventilation procedure. For example, a user may indicate (e.g., by selecting from a menu, touching, typing, or otherwise providing input) that a recruitability assessment is desired. Responsive to such an indication, the user interface for specifying ventilation procedure 602 may be displayed.

In some examples, prior to displaying the interface shown on display 600 and/or prior to initiating a recruitability assessment, one or more warnings may be displayed on display 600 (e.g., as specified by executable instructions for automated ventilation procedure(s) 112). The warnings prior to initiating a recruitability assessment may include, for example, warning that the procedure may cause barotrauma and/or hemodynamic instability. The warning may recommend ensuring suitability of the procedure. A warning may be provided in some examples to ensure the patient is apneic during the procedure. In some examples, while the ventilator may, e.g., utilizing trigger settings, detect patient efforts for respiration and may provide an alert of patient efforts, the ventilator may not deliver triggered breaths (e.g., triggered from patient effort) during the recruitability assessment. A warning may be provided that the recruitability assessment is available only for certain modes—e.g., A/CMV PCV or SIMV PCV in some examples. A warning may be displayed regarding the use of incremental and decremental steps in the recruitability assessment and against manually changing settings during the automated assessment. Initial instructions may be displayed on display 600 in some examples for a user—e.g., to select an appropriate mode of the ventilator for the recruitability assessment, and/or to set one or more appropriate initial ventilation parameters (e.g., set PEEP and/or ΔPC (or PLIMIT) appropriate for a patient and in such a way that ΔPC or inflation pressure (e.g., PUMIT-PEEP) is between 10-20 cmH2O in some examples.

The user interface for specifying ventilation procedure 602 includes prompts for a user to enter ventilation parameters which may be used in a recruitability assessment -maximum plateau pressure, PEEP change per step, and duration for each step. The user interface for specifying ventilation procedure 602 further includes a button for a user to start the procedure.

Examples of entered user values are shown in FIG. 6—45 cmH₂O maximum plateau pressure, 5 cmH₂O PEEP change each step, and 30 second duration for each PEEP step. Other values may be used in other examples. In some examples, the executable instructions for automated ventilation procedure(s) 112 may enforce certain ranges for one or more of the parameters. For example, maximum plateau pressure may be enforced within a range of 30-55 cmH2O. PEEP step increment may be enforced to be within a range of 2-5 cmH₂O. Step duration may be enforced to be within a range of 20-120 sec. PEEP after the maneuver may be enforced to be within a range of 0-30 cmH₂O. If a user attempts to enter a value for a parameter outside the pre-determined range (note ranges may be stored, for example in memory 110 of FIG. 1), then the display 600 may display an alert regarding the out-of-range parameter, or the parameter may in some other way be rejected by the system. In some examples, parameter values on the display 600 may default to predetermined values. For example, maximum plateau pressure may have a default value of 40 cmH₂O. PEEP step may have a default value of 5 cmH₂O. PEEP step duration may have a default value of 30 seconds. The default values may be stored, for example, in memory 110 of FIG. 1). The default values may be displayed on display 600 when a user initially selects to perform a recruitability assessment. The user may then need only change the parameters for which the defaults are not desired. Other ranges and/or default values may be used in other examples. If a user attempts to initiate the recruitability assessment without a prerequisite being met (e.g., appropriate ventilation mode, appropriate initial settings, in-range ventilation parameters provided), the system may provide an alert and may not initiate the recruitability assessment.

Once a user enters the requested ventilation parameters in through the user interface (e.g., by typing, touching, entering, gesturing, speaking, or otherwise providing input), the ventilation parameters may be stored, e.g., as ventilation parameter(s) 126 in FIG. 1. The input ventilation parameters may then be used to perform the recruitability assessment when the user indicates to begin (e.g., by clicking, touching or otherwise providing a start input).

As shown in FIG. 6, the display 600 may include a ventilation procedure preview 604. The preview may provide a graphical view of the expected actions of the ventilator responsive to the entered ventilation parameters.

Generally, examples described herein may provide a recruitability assessment by providing tidal breaths to a patient beginning at a starting PEEP pressure for a step duration specified by the user. After the step duration, the PEEP pressure may be incremented by a PEEP increment specified by a user to obtain a next pressure. Tidal breaths at the next pressure may be provided to a patient for the step duration specified for the user. This process may be repeated until the maximum plateau pressure specified by the user is attained.

Once the recruitability assessment has attained the maximum pressure specified by the user, the recruitability assessment may decrement the PEEP pressure by the PEEP increment to obtain a next pressure. Tidal breaths at the next pressure may be provided for the step duration. This may be completed until the starting PEEP pressure is again reached. After returning to the starting PEEP pressure, the recruitability assessment may be completed.

The ventilation procedure preview 604 displays a graph of pressure vs. time that displays the expected ventilator performance for the maneuver. The ventilation procedure preview 604 may display the expected ventilation procedure using the ventilation parameters entered by a user in user interface for specifying ventilation procedure 602. A user may graphically review this performance prior to initiating the automated procedure (e.g., prior to pressing ‘start’). In some examples, the user may revise the parameters entered in the user interface for specifying ventilation procedure 602 after review of the ventilation procedure preview 604.

In some examples, the executable instructions for automated ventilation procedures described herein may include instructions for providing one or more alarms. The ventilation procedure preview 604 shown in FIG. 6 illustrates an example of an alarm condition. For example, an alarm level 606 may be set such that if a pressure exceeds the alarm level 606, the ventilator and/or computing system will provide an alarm (e.g., an audible, visual, and/or tactile alarm). The alarm level 606 may be displayed in the ventilation procedure preview 604. In some examples, the alarm level 606 may be set by a user, however in some examples the alarm level 606 may be specified by the executable instructions for automated ventilation procedure(s) themselves. Alarms may provide additional safety in some examples.

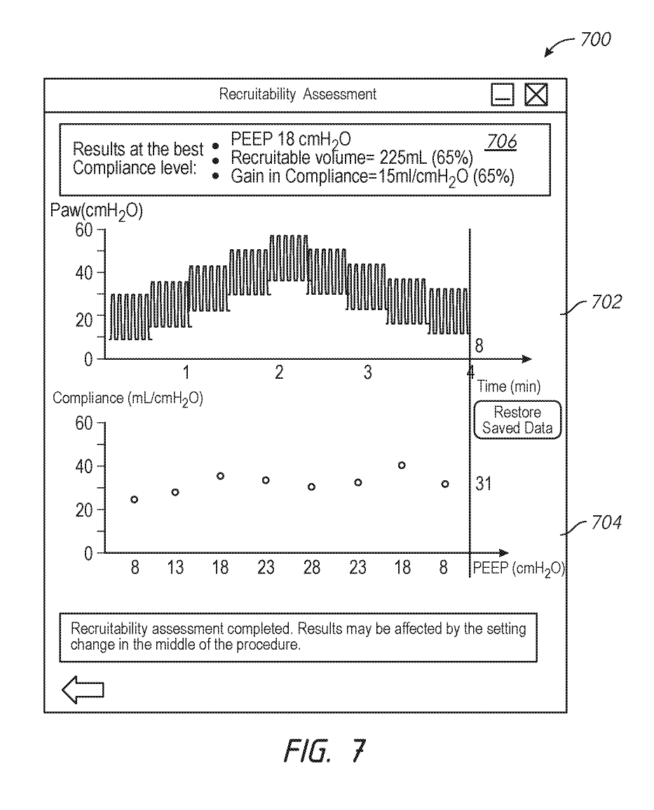

FIG. 7 is a schematic illustration of a display arranged in accordance with examples described herein. Display 700 is shown displaying ventilator data 702, patient data 704, and results 706. In other examples, additional, fewer, and/or different elements may be displayed. The display 700 may be used to implement and/or may be implemented by display 114, display 120, and/or display 600 in some examples. Display 700 is shown displaying information that executable instructions described herein (e.g., executable instructions for automated ventilation procedure(s) 112 of FIG. 1) may cause to display during and/or after a recruitability assessment.

During an automated ventilation procedure, such as a recruitability assessment, display 700 may display ventilator data 702—e.g., data regarding one or more ventilator parameters. Data may be displayed as the ventilator performs the procedure, and may provide information regarding progress of the procedure (e.g., the data may be displayed in real-time). For example, as shown in FIG. 7, a graph of pressure over time is depicted as ventilator data 702. The graph may be dynamic in the display 700 in that it is drawn as the procedure progresses, so that a user may view how the procedure is progressing. While a graph is shown, any of a variety of data display types may be used (e.g., line graph, bar graph, pie graph, text list). Generally, one or more correlations between ventilator data and time or other parameter may be displayed.

During the automated ventilation procedure, such as a recruitability assessment, display 700 may additionally or instead display patient data 704—e.g., data regarding one or more patient parameters. Any of a variety of patient parameters may be displayed, including, but not limited to lung compliance, ventilation volume, blood oxygen saturation, transpulmonary pressure, esophageal pressure, or combinations thereof. In FIG. 7, the display 700 is shown displaying a graph of lung compliance vs. PEEP pressure. The measured parameter (e.g., lung compliance) may similarly appear in the display 700 dynamically, such that the graph is drawn over time (e.g., in real-time) and a user may view how the patient parameter is progressing during the procedure. While not shown in FIG. 7, historical patient data may also be displayed during recruitability assessments described herein

In some examples, ventilator data 702 and patient data 704 may be depicted on a same display, e.g., display 700, during an automated ventilation procedure. This may advantageously allow a user to review both the progress of the automated ventilation procedure and the patient parameter.

During the automated ventilation procedure (e.g., the recruitability assessment), the system (e.g., system 100 in accordance with executable instructions for automated ventilation procedure(s) 112) may provide an alert if a patient's own respiratory efforts are detected. The alert may be provided, for example, by computing system 106 and/or ventilator 102 of FIG. 1. The alert may be audible, visual, and/or tactile in some example. The alert may be provided, for example, on display 700 and/or display 114 and/or display 120. In some examples, the system (e.g., computing system 106 of FIG. 1) may halt the automated ventilation procedure responsive to an alert of a patient's respiratory efforts.

In some examples, the display 700 may display one or more results or other data analysis outputs, e.g., results 706. For example, the executable instructions for automated ventilation procedure(s) 112 may include instructions for analyzing data generated during one or more ventilation procedures, such as a recruitability assessment. In the example of FIG. 7, the results 706 reported include a PEEP at the highest patient compliance value during the recruitability assessment—18 cmH₂O in the example of FIG. 7. The results 706 also include a recruitable volume estimated for the patient (e.g., 225 mL, in the example of FIG. 7) and a gain in compliance expected (e.g., 15 mLcmH₂O in the example of FIG. 7). In this manner, a PEEP at which a patient has a highest lung compliance, and an efficacy of a recruitment maneuver, may be reported in an automated manner. Generally, in examples described herein, results may include one or more ventilation parameters (e.g., PEEP) reported at a particular patient criteria (e.g., highest lunch compliance, a lowest transpulmonary pressure, a highest oxygen saturation, or combinations thereof during the positive end-expiratory pressure titration). As described with reference to FIG. 6 and FIG. 7, a user may enter ventilator parameters, start the automated procedure, and then be presented with a result, such as the PEEP at which the patient has the highest lung compliance. The result may be used in a number of ways, including for subsequent treatment of a patient. For example, the PEEP reported by results 706 may be used to ventilate the patient after the recruitability assessment is complete. The recruitable volume and/or compliance gain results may be used to determine whether or not to perform a recruitment maneuver.

While various aspects and embodiments have been disclosed herein, other aspects and embodiments are contemplated. The various aspects and embodiments disclosed herein are for purposes of illustration and are not intended to be limiting.

1. A system comprising:
a ventilator; and
a computing system comprising:
a display;
a processor coupled to the display; and
a memory encoded with executable instructions for performing an automated ventilation procedure, wherein the executable instructions, when executed by the processor, cause the display to display a user interface for entry of ventilation parameters, cause the computing system to receive entered ventilation parameters, cause the ventilator to perform the automated ventilation procedure using the entered ventilation parameters, and cause the ventilator, the display, or both to display, during at least a portion of the automated ventilation procedure, a correlation between positive end-expiratory pressure (PEEP) provided by the ventilator during the automated ventilation procedure and at least one other patient parameter.

2. The system of claim 1, wherein the at least one other patient parameter comprises lung compliance, ventilation volume, airway pressure, blood oxygen saturation, transpulmonary pressure, esophageal pressure, a circulatory parameter, or combinations thereof.

3. The system of claim 1, wherein the automated ventilation procedure comprises a recruitment maneuver, a PEEP titration, a recruitability assessment, or combinations thereof.

4. The system of claim 1, wherein the executable instructions further cause the ventilator to automatically transition from a recruitment maneuver to a positive end-expiratory pressure titration using at least one result from the recruitment maneuver.

5. The system of claim 1, wherein the executable instructions further cause the display to display a predicted time course of PEEP based on the entered ventilation parameters prior to beginning the automated ventilation procedure.

6. A method comprising:
display a user interface for entry of parameters for an automated recruitability assessment, the parameters including maximum pressure, pressure change per step, and step duration;
receive selected values for the maximum pressure, pressure change per step, and step duration;
control a ventilator to perform the automated recruitability assessment on a patient using the selected values, wherein the automated recruitability assessment comprises:
providing tidal cycles of breaths at an initial pressure for the step duration;
increasing the initial pressure by the selected value for pressure change per step to obtain a next pressure and providing a tidal cycles of breaths at the next pressure for another step duration;
repeating the providing and increasing until the selected value for the maximum pressure is reached;
decreasing the maximum pressure by the selected value for pressure change per step to obtain a next decreased pressure and providing tidal cycles of breaths at the next decreased pressure for the step duration; and
repeating the decreasing and providing until the initial pressure is reached; and
displaying an estimated recruitable value based on the automated recruitability assessment.

7. The method of claim 6, further comprising, prior to said control the ventilator, displaying a predicted time course of positive end-expiratory pressure (PEEP) based on the selected values.

8. The method of claim 6, further comprising, during said control the ventilator, displaying real-time correlations between PEEP and at least one patient parameter.

9. The method of claim 8, wherein the at least one patient parameter comprises lung compliance, ventilation volume, blood oxygen saturation, transpulmonary pressure, esophageal pressure, or combinations thereof.

10. The method of claim 6, further comprising, providing an alert responsive to detection of a patient's respiratory efforts during the automated recruitability assessment.

11. The method of claim 6, further comprising providing an alert responsive to at least one of the selected values being outside of a predetermined range for the automated recruitability assessment.

12. The method of claim 6, wherein a PEEP at a particular patient criteria is automatically reported after the automated recruitability assessment.

13. The method of claim 12, wherein the particular patient criteria comprises a highest lung compliance, a lowest transpulmonary pressure, and/or a highest oxygen saturation during the recruitability assessment.

14. The method of claim 6, further comprising displaying historical patient data of PEEP from a previously-performed ventilation procedure during the performance of the automated recruitability assessment.

15. The method of claim 6, further comprising, at least in part during said control the ventilator, display a correlation between positive end-expiratory pressure and at least one other patient parameter.

16. The method of claim 15, wherein the at least one other patient parameter comprises lung compliance, ventilation volume, blood oxygen saturation, transpulmonary pressure, esophageal pressure, or combinations thereof.

17. A method comprising:
display a user interface for entry of parameters for a positive end-expiratory pressure titration, including positive end-expiratory pressure decrement, minimum positive end-expiratory pressure, and step duration;
control a ventilator to automatically perform the positive end-expiratory pressure titration using the parameters; and
receive a reported positive end-expiratory pressure at a particular patient criteria.

18. The method of claim 17, wherein the particular patient criteria comprises a highest lung compliance, a lowest transpulmonary pressure, a highest oxygen saturation, or combinations thereof during the positive end-expiratory pressure titration.

19. The method of claim 17, further comprising displaying historical patient data comprising correlations between the positive end-expiratory pressure titration and the parameters.

20. The method of claim 17, wherein, at least in pail during said control the ventilator, display a current correlation between the positive end-expiratory pressure and at least one other patient parameter.
